(19) United States
(12) Patent Application Publication
(10) Pub. No.: US 2010/0196935 A1
(43) Pub. Date: Aug. 5, 2010

(54) TREATING PRE-ECLEMPSIA AND CARDIOVASCULAR DISEASES

(75) Inventors: Vijayalakshmi Shridhar, Rochester, MN (US); Jeremy R. Chien, Rochester, MN (US); Brian C. Brost, Rochester, MN (US); Funminiyi A. Ajayi, Rochester, MN (US)

Correspondence Address: FISH & RICHARDSON P.C., PO BOX 1022, MINNEAPOLIS, MN, 55440-1022, US

(73) Assignee: MAYO FOUNDATION FOR MEDICAL EDUCATION AND RESEARCH, Rochester, MN (US)

(21) Appl. No.: 12/530,917

(22) PCT Filed: Mar. 12, 2008
(86) PCT No.: PCT/US2008/056707

§ 371 Date: Apr. 6, 2010

Related U.S. Application Data
(60) Provisional application No. 60/906,622, filed on Mar. 12, 2007.

(51) Int. Cl. G01N 33/53; G01N 33/00

(52) U.S. Cl. 435/7.92; 436/86

(57) ABSTRACT

This document relates to methods and materials involved in identifying, predicting, and treating pre-eclampsia or a cardiovascular disease (e.g., atherosclerosis) in mammals. For example, methods and materials involved in using serum levels of HtrA polypeptides to identify pre-eclampsia or a cardiovascular disease, using HTRA polymorphisms to predict pre-eclampsia or a cardiovascular disease, and using HtrA antibodies to treat pre-eclampsia or a cardiovascular disease in mammals (e.g., humans) are provided.

### BACKGROUND

1. Technical Field

This document relates to methods and materials involved in using HtrA polypeptides, polymorphisms, and antibodies to identify, predict, and treat pre-eclampsia and cardiovascular diseases (e.g., atherosclerosis and acute coronary syndrome) in mammals. For example, this document relates to methods and materials involved in using elevated serum levels of HtrA polypeptides to identify pre-eclampsia or a cardiovascular disease, using the presence of one or more HTRA polymorphisms to predict pre-eclampsia or a cardiovascular disease, and using HtrA antibodies to treat pre-eclampsia or a cardiovascular disease.

2. Background Information

The HtrA (high temperature requirement A) polypeptide family is a highly conserved family of serine proteases, which can be characterized by the combination of a trypsin-like catalytic domain with at least one C-terminal PDZ domain. A bacterial HtrA polypeptide was identified in E. coli as a periplasmic protein required for high temperature tolerance. This bacterial HtrA polypeptide is reported to have a molecular chaperone activity at low temperatures and a serine protease activity that degrades mis-folded proteins at high temperatures. The human HtrA family of proteases includes HtrA1, HtrA2, HtrA3, and HtrA4 polypeptides.

### SUMMARY

This document provides methods and materials involved in using HtrA polypeptides, polymorphisms, and antibodies to identify, predict, and treat pre-eclampsia or a cardiovascular disease (e.g., atherosclerosis and acute coronary syndrome) in mammals. For example, this document relates to methods and materials involved in using elevated serum levels of HtrA polypeptides to identify pre-eclampsia or a cardiovascular disease, using the presence of one or more HTRA polymorphisms to predict pre-eclampsia or a cardiovascular disease, and using HtrA antibodies to treat pre-eclampsia or a cardiovascular disease. The methods and materials provided herein can allow clinicians to diagnose pre-eclampsia or cardiovascular diseases at earlier stages and reduce death and disability associated with these diseases.

In general, one aspect of this document features a method of identifying pre-eclampsia in a mammal. The method comprises, or consists essentially of, (a) determining whether or not a mammal contains an elevated level or activity of an HtrA polypeptide, and (b) classifying the mammal as having pre-eclampsia when the elevated level or activity is present. The mammal can be a human. The HtrA polypeptide can be an HtrA1 polypeptide. The level or activity can be a serum level or activity. The determining step can comprise using a radioimmunoassay, an ELISA, or a proteolytic colorimetric assay.

In another aspect, this document features a method of identifying a cardiovascular disease in a mammal. The method comprises, or consists essentially of, (a) determining whether or not a mammal contains an elevated level or activity of an HtrA polypeptide, and (b) classifying the mammal as having a cardiovascular disease when the elevated level or activity is present. The mammal can be a human. The cardiovascular disease can be atherosclerosis or acute coronary syndrome. The HtrA polypeptide can be an HtrA1 polypeptide. The level or activity can be a serum level or activity. The determining step can comprise using a radioimmunoassay, an ELISA, or a proteolytic colorimetric assay.

In another aspect, this document features a method of predicting the susceptibility of a mammal to develop pre-eclampsia. The method comprises, or consists essentially of, (a) determining whether or not a mammal has an HtrA polymorphism, and (b) classifying the mammal as being susceptible to develop pre-eclampsia when the HtrA polymorphism is present. The mammal can be a human. The HtrA polymorphism can be an HtrA1 polymorphism. The HtrA1 polymorphism can be the single nucleotide polymorphism rs11200638.

In another aspect, this document features a method of predicting the susceptibility of a mammal to develop a cardiovascular disease. The method comprises, or consists essentially of, (a) determining whether or not a mammal has an HtrA polymorphism, and (b) classifying the mammal as being susceptible to develop a cardiovascular disease when the HtrA polymorphism is present. The mammal can be a human. The cardiovascular disease can be atherosclerosis or acute coronary syndrome. The HtrA polymorphism can be an HtrA1 polymorphism. The HtrA1 polymorphism can be the single nucleotide polymorphism rs11200638.

In another aspect, this document features a method of treating pre-eclampsia in a mammal. The method comprises, or consists essentially of, administering an HtrA antibody to a mammal having pre-eclampsia under conditions wherein the severity or frequency of a symptom of the pre-eclampsia is reduced. The mammal can be a human. The HtrA antibody can be a monoclonal HtrA antibody. The HtrA antibody can be an HtrA1 antibody. The HtrA antibody can be capable of inhibiting proteolytic activity of an HtrA polypeptide. The method can comprise identifying the mammal as having the pre-eclampsia prior to the administering step.

In another aspect, this document features a method of treating a cardiovascular disease in a mammal. The method comprises, or consists essentially of, administering an HtrA antibody to a mammal having a cardiovascular disease under conditions wherein the severity or frequency of a symptom of the cardiovascular disease is reduced. The mammal can be a human. The HtrA antibody can be a monoclonal HtrA antibody. The HtrA antibody can be an HtrA1 antibody. The HtrA antibody can be capable of inhibiting proteolytic activity of an HtrA polypeptide. The cardiovascular disease can be atherosclerosis or acute coronary syndrome. The method can comprise identifying the mammal as having the cardiovascular disease prior to the administering step.

In another aspect, this document features a method of treating pre-eclampsia in a mammal. The method comprises, or consists essentially of, administering an HtrA polypeptide to a mammal having pre-eclampsia under conditions wherein the mammal produces HtrA antibodies. The method can comprise identifying the mammal as having the pre-eclampsia prior to the administering step.

In another aspect, this document features a method of treating a cardiovascular disease in a mammal. The method comprises, or consists essentially of, administering an HtrA polypeptide to a mammal having a cardiovascular disease under conditions wherein the mammal produces HtrA antibodies. The method can comprise identifying the mammal as having the cardiovascular disease prior to the administering step. The cardiovascular disease can be atherosclerosis or acute coronary syndrome.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1A is a Western blot analyzing lysates of JEG-3 cells that were transiently transfected with a vector containing a nucleic acid encoding a GFP polypeptide (GFP), a wild-type HtrA1 polypeptide (HtrA1(WT)), or a protease mutant HtrA1 polypeptide (HtrA1 (SA)). Multiple bands below 45 kD in lysates from cells transfected with a vector containing a nucleic acid encoding a wild-type HtrA1 polypeptide represent autoproteolysis of HtrA1 polypeptide. The lower panel is a loading control that was immunoblotted with anti β-actin antibodies. FIGS. 1B and 1C contain fluorescence photomicrographs of JEG-3 cells that were permeabilized (panel B) or unpermeabilized (panel C) and stained using antibodies against HtrA1 or F-actin polypeptide or using Hoechst dye.

FIG. 2 contains photomicrographs of propidium iodide-stained migrated cells transfected with a vector containing a nucleic acid encoding a GFP polypeptide (panel A), an SA HtrA1 polypeptide (panel B), or a WT HtrA1 polypeptide (panel C). FIG. 2D is a graph plotting the number of cells migrated per microscopic field for cells transfected with a vector containing a nucleic acid encoding a GFP, SA HtrA1, or WT HtrA1 polypeptide. Bar=Mean±s.e.m., **=P<0.01, α=0.05.

FIG. 3A contains photomicrographs of cells transfected with a vector containing a nucleic acid encoding a GFP, SA HtrA1, or WT HtrA1 polypeptide. The photomicrographs were taken 0, 16, or 48 hours after a scratch wound was made. FIG. 3B is a graph plotting percentage of the gap filled versus time after a scratch wound was made. Bar=Mean±s.e.m., **=P<0.01, α=0.05.

FIG. 4 contains photomicrographs of propidium iodide-stained invaded cells that were transfected with a vector containing a nucleic acid encoding a GFP polypeptide (panel A), a SA HtrA1 polypeptide (panel B), or a WT HtrA1 polypeptide (panel C). FIG. 4D is a graph plotting number of invaded cells per microscopic field for cells transfected with a vector containing a nucleic acid encoding a GFP, SA HtrA1, or WT HtrA1 polypeptide. Bar=Mean±s.e.m., **=P<0.01, α=0.05.

FIG. 5A is a graph plotting percent invasion of JEG-3 cells transfected with a vector containing a nucleic acid encoding a GFP, SA HtrA1, or WT HtrA1 polypeptide. FIG. 5B is a graph plotting invasion index of JEG-3 cells transfected with a vector containing a nucleic acid encoding a GFP, SA HtrA1, or WT HtrA1 polypeptide.

FIG. 6A is a photomicrograph of placental tissue from pregnancy complicated by pre-eclampsia. FIG. 6B is a photomicrograph of placental tissue from normal pregnancy. FIG. 6C is a graph plotting HtrA1 polypeptide expression in villous trophoblasts from normal and severe pre-eclamptic pregnancies at all gestational ages studied. FIG. 6D is a graph plotting HtrA1 polypeptide expression in villous trophoblasts from normal and severe pre-eclamptic pregnancies at gestational age >34 weeks. FIG. 6E is a graph plotting HtrA1 polypeptide expression in villous trophoblasts from normal and severe pre-eclamptic pregnancies at gestational age <34 weeks. FIG. 6F is a graph plotting HtrA1 polypeptide expression in extravillous trophoblasts from normal and severe pre-eclamptic pregnancies at gestational age <34 weeks.

FIG. 7 is a graph plotting % B/B0 versus amount of HtrA1 polypeptide measured using a radioimmunoassay.

FIG. 8 contains photomicrographs of atherosclerotic plaques stained with H&E, anti-HtrA1 antibodies, or control IgG antibodies.

FIG. 9A is a Northern blot analyzing HtrA1 RNA expression in various tissues. FIG. 9B is a graph plotting results in absorbance at 450 nm obtained by measuring circulating HtrA1 levels in control and myocardial injury patients using an ELISA assay.

FIG. 10 is a Western blot analyzing HtrA1 polypeptide expression in OSEtsT cells following exposure to the indicated agents. The middle panel is a loading control that was immunoblotted with anti β-actin antibodies. The lower panel is a graph plotting results of densitometric analysis of HtrA1 polypeptide expression.

FIG. 11 is a graph plotting the absorbance at 450 nm for the indicated amounts of HtrA1 polypeptides.

FIG. 12 is a graph plotting the absorbance at 450 nm for the indicated amounts of HtrA1 polypeptides using a sandwich ELISA.

### DETAILED DESCRIPTION

This document provides methods and materials related to identifying, predicting, and treating pre-eclampsia and cardiovascular diseases (e.g., atherosclerosis and acute coronary syndrome) in mammals. For example, this document provides methods and materials for identifying a mammal as having pre-eclampsia or a cardiovascular disease based on an elevated level or activity of an HtrA polypeptide in the mammal (e.g., in the serum of the mammal). This document also provides methods and materials for predicting whether or not a mammal is susceptible to developing pre-eclampsia or a cardiovascular disease based on the presence of an HtrA polymorphism in the mammal. In addition, this document provides methods and materials for treating pre-eclampsia or a cardiovascular disease in a mammal using an HtrA polypeptide or antibody directed against an HtrA polypeptide.

Pre-eclampsia or a cardiovascular disease can be identified, predicted, or treated in any mammal (e.g., human, dog, cat, horse, and cow). In some cases, pre-eclampsia or a cardiovascular disease can be identified, predicted, or treated in an elderly, diabetic, or overweight human, or a human with a family history of pre-eclampsia or a cardiovascular disease. In some cases, pre-eclampsia or a cardiovascular disease can be identified, predicted, or treated in a human that is a smoker, is hypertensive, has hypercholesterolemia, or has used estrogen postmenopausally. Examples of cardiovascular diseases include, without limitation, atherosclerosis, coronary artery disease, heart attack, myocardial infarction, angina, congestive heart failure, hardening of the arteries, stroke, or peripheral vascular disease.

As disclosed herein, a mammal can be identified as having pre-eclampsia or a cardiovascular disease (e.g., atherosclerosis or acute coronary syndrome) if the level or activity of an HtrA polypeptide in the mammal (e.g., in a serum sample from the mammal) is an elevated level. If the level or activity of an HtrA polypeptide in a mammal (e.g., in a serum sample from the mammal) is not an elevated level, then the mammal can be classified as not having pre-eclampsia or a cardiovascular disease.

An HtrA polypeptide can be any polypeptide having HtrA polypeptide activity. For example, an HtrA polypeptide can belong to the HtrA polypeptide family. In some cases, an HtrA polypeptide can be an HtrA1, HtrA2, HtrA3, or HtrA4 polypeptide. In some cases, an HtrA polypeptide can be a homolog or an ortholog of an HtrA polypeptide or can be a human HtrA polypeptide having one or more amino acid changes.

Examples of HtrA polypeptides include, without limitation, human HtrA polypeptides set forth in GenBank gi number 4506141 (GenBank Accession No. NP_{—}002766), 116283290 (GenBank Accession No. AAH11352), 7019477 (GenBank Accession No. NP_{—}037379), 21614538 (GenBank Accession No. NP_{—}659540), 22129776 (GenBank Accession No. NP_{—}444272), and 24308541 (GenBank Accession No. NP_{—}710159). Additional examples of HtrA polypeptides include, without limitation, mouse HtrA serine peptidase 1 set forth in GenBank gi number 15488756 (GenBank Accession No. AAH13516), mouse HtrA serine peptidase 2 set forth in GenBank gi number 29437202 (GenBank Accession No. AAH49880), mouse HtrA serine peptidase 3 isoform a precursor set forth in GenBank gi number 110815869 (GenBank Accession No. NP_{—}084403), mouse HtrA serine peptidase 4 set forth in GenBank gi number 124487143 (GenBank Accession No. NP_{—}001074656), and rat HtrA serine peptidase 1 set forth in GenBank gi number 51859442 (GenBank Accession No. AAH81767).

The term “elevated level” as used herein with respect to the level or activity of an HtrA polypeptide is any level or activity that is greater than a reference level or activity, respectively, for an HtrA polypeptide. The term “reference level” as used herein with respect to an HtrA polypeptide level or activity is the level or activity, respectively, of an HtrA polypeptide typically found in mammals free of pre-eclampsia and a cardiovascular disease. For example, a reference level or activity of an HtrA polypeptide can be the average level or activity, respectively, of HtrA polypeptide that is present in samples obtained from a random sampling of 50 healthy mammals (e.g., humans). It will be appreciated that levels from comparable samples are used when determining whether or not a particular level is an elevated level.

An elevated level or activity of an HtrA polypeptide can be any level or activity provided that the level or activity is greater than a corresponding reference level or activity, respectively, for an HtrA polypeptide. For example, an elevated level or activity of an HtrA1 polypeptide can be 0.5, 1, 2, 3, 4, 5, 6, 7, 8, 9, 10, 15, 20, or more times greater than the reference level or activity, respectively, for an HtrA1 polypeptide. In addition, a reference level or activity can be any amount. For example, a reference level for an HtrA1 polypeptide can be zero. In this case, any level of HtrA1 polypeptide greater than zero would be an elevated level.

Any method can be used to determine the level or activity of an HtrA polypeptide present within a sample. For example, anti-HtrA polypeptide antibodies can be used to determine the level of HtrA polypeptide expression within a sample. In some embodiments, the level of an HtrA polypeptide present within a sample can be determined using polypeptide detection methods such as Western blot and immunochemistry techniques (e.g., a sandwich ELISA technique). A method that can be used to determine the activity of an HtrA polypeptide present within a sample can be a proteolytic colorimetric assay, such as that described in Example 3.

The level of an HtrA polypeptide present within a sample also can be determined by measuring the level of an mRNA that encodes an HtrA polypeptide. Any method can be used to measure the level of an RNA encoding an HtrA polypeptide including, without limitation, PCR-based methods. For example, quantitative PCR or RT-PCR can be used with oligonucleotide primers designed to amplify nucleic acid (e.g., RNA) encoding an HtrA polypeptide. Any method can be used to identify primers capable of amplifying nucleic acid encoding an HtrA polypeptide. For example, a computer algorithm can be used to search a database (e.g., GenBank®) for HtrA nucleic acid. Any method can be used to analyze the amplified products. For example, amplified products corresponding to HtrA mRNA can be separated by gel electrophoresis, and the level of HtrA-specific product determined by densitometry. In some cases, the level of HtrA-specific product can be determined by quantitative RT-PCR using fluorescent beacons or dyes.

Any type of sample can be used to evaluate the level or activity of an HtrA polypeptide including, without limitation, serum, blood, and plasma. In addition, any method can be used to obtain a sample. For example, a blood sample can be obtained by peripheral venipuncture. Once obtained, a sample can be manipulated prior to measuring the level or activity of an HtrA polypeptide. For example, a blood sample can be treated such that total mRNA is obtained. Once obtained, the total mRNA can be evaluated to determine the level of HtrA mRNA present. In another example, a blood sample can be disrupted to obtain a cell lysate. Once obtained, the cell lysate can be analyzed using anti-HtrA polypeptide antibodies (e.g., anti-HtrA1 polypeptide antibodies) to determine the level of HtrA polypeptide (e.g., HtrA1 polypeptide) present within the sample.

This document also provides methods and materials to assist medical or research professionals in determining whether or not a mammal has pre-eclampsia or a cardiovascular disease. Medical professionals can be, for example, doctors, nurses, medical laboratory technologists, and pharmacists. Research professionals can be, for example, principle investigators, research technicians, postdoctoral trainees, and graduate students. A professional can be assisted by (1) determining the level of an HtrA polypeptide in a sample, and (2) communicating information about that level to that professional.

Any method can be used to communicate information to another person (e.g., a professional). For example, information can be given directly or indirectly to a professional. In addition, any type of communication can be used to communicate the information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional.

In addition to identifying pre-eclampsia or a cardiovascular disease in mammals, methods and materials provided herein can be used to determine whether or not a mammal is susceptible to develop pre-eclampsia and a cardiovascular disease (e.g., atherosclerosis). For example, methods and materials are provided herein for determining whether or not a sample (e.g., a genomic DNA sample) from a mammal contains a polymorphism in an HTRA nucleic acid. The term “HTRA nucleic acid” as used herein refers to any nucleic acid that encodes an HtrA polypeptide, or any fragment of such a nucleic acid. Examples of HTRA nucleic acids include, without limitation, the nucleic acid sequences set forth in GenBank gi numbers 73747816 (GenBank Accession No. NM_{—}002775) and 15030191 (GenBank Accession No. BC011352).

The methods and materials provided herein can be used to determine whether or not an HTRA nucleic acid of a mammal (e.g., human) contains a polymorphism, such as a single nucleotide polymorphism (SNP). For example, methods and materials provided herein can be used to determine whether a mammal has an re11200638 SNP (Yang et al., Science, 314:992 (2006)). Any method can be used to detect a polymorphism in an HTRA nucleic acid. For example, polymorphisms can be detected by sequencing exons, introns, or untranslated sequences, denaturing high performance liquid chromatography (DHPLC), allele-specific hybridization, allele-specific restriction digests, mutation specific polymerase chain reactions, single-stranded conformational polymorphism detection, and combinations of such methods.

In some embodiments, genomic DNA can be used to detect HTRA polymorphisms. Genomic DNA is typically extracted from a biological sample, such as a peripheral blood sample or a tissue sample. Standard methods can be used to extract genomic DNA from a biological sample, such as phenol extraction. In some cases, genomic DNA can be extracted using a commercially available kit (e.g., from Qiagen, Chatsworth, Calif.; Promega, Madison, Wis.; or Gentra Systems, Minneapolis, Minn.).

A mammal containing one or more polymorphisms in an HTRA nucleic acid can be classified as being susceptible to develop pre-eclampsia or a cardiovascular disease (e.g., atherosclerosis) as compared to a corresponding mammal containing wild-type HTRA nucleic acid at both alleles.

Mammals identified as having pre-eclampsia or a cardiovascular disease (e.g., atherosclerosis) or being susceptible to develop pre-eclampsia or a cardiovascular disease can be treated by administering an HtrA polypeptide or antibody against an HtrA polypeptide. As described herein, pre-eclampsia or a cardiovascular disease can be treated using an HtrA polypeptide or an antibody directed against an HtrA polypeptide. Administering an HtrA polypeptide or antibody to a mammal can reduce the severity or frequency of one or more symptoms of pre-eclampsia or the cardiovascular disease in the mammal. For example, administering an HtrA polypeptide or antibody to a mammal can reduce the severity or frequency of chest pain, heart rhythms, heart failure, stroke, heart attacks, leg cramps, high blood pressure, or kidney failure. In some cases, administering an HtrA polypeptide or antibody to a mammal can reduce loss of vision, bleeding of an abnormal blood vessel, distortion of vision, or difficulty reading or watching television. The effect of administering an HtrA polypeptide or antibody on a symptom of pre-eclampsia or a cardiovascular disease can be of any magnitude. For example, administering an HtrA polypeptide or antibody can reduce the severity or frequency of a symptom of pre-eclampsia or a cardiovascular disease by 1%, 2%, 3%, 4%, 5%, 7.5%, 10%, 15%, 20%, 25%, 30%, 35%, 40%, 45%, 50%, or more.

Administering an HtrA polypeptide to a mammal can be designed to produce HtrA antibodies in the mammal. For example, an HtrA polypeptide that is foreign to a mammal's immune system can be administered to the mammal so that the mammal produces HtrA antibodies that can inhibit the protease activity of an HtrA polypeptide in the mammal. In some cases, a self HtrA polypeptide can be designed to contain foreign T-cell epitopes so that administration of the polypeptide produces HtrA antibodies that can inhibit the protease activity of an HtrA polypeptide in the mammal. Adjuvants such as alum can be used in combination with HtrA polypeptides. The protease activity can be inhibited by any amount. For example, the protease activity can be inhibited by 5%, 10%, 20%, 30%, 40%, 50%, 55%, 60%, 70%, 75%, 80%, 95%, or 100%.

An HtrA1 polypeptide can be a recombinant HtrA polypeptide, a synthetic HtrA polypeptide, an isolated HtrA polypeptide, a purified HtrA polypeptide, or a commercially available HtrA polypeptide. An HtrA polypeptide can have a non-naturally occurring sequence or can have a sequence present in any species (e.g., human, rat, or mouse). In some cases, an HtrA polypeptide can contain one or more amino acid analogs or other peptidomimetics. The subunits of an HtrA polypeptide may be linked by peptide bonds or other bonds such as, for example, ester or ether bonds. An HtrA polypeptide can be a full-length HtrA polypeptide, a precursor HtrA polypeptide, or a fragment of an HtrA polypeptide. In some cases, an HtrA polypeptide can contain one or more modifications. For example, an HtrA polypeptide can be modified to be pegylated or to contain additional amino acid sequences such as an albumin sequence (e.g., a human albumin sequence). In some cases, an HtrA polypeptide can be a fusion polypeptide, such as a fusion polypeptide that contains a fragment of an albumin sequence. In some cases, an HtrA polypeptide can be covalently attached to oligomers, such as short, amphiphilic oligomers that enable oral administration or improve the pharmacokinetic or pharmacodynamic profile of a conjugated HtrA polypeptide. The oligomers can comprise water soluble polyethylene glycol (PEG) and lipid soluble alkyls (short chain fatty acid polymers). See, for example, International Patent Application Publication No. WO 2004/047871. In some cases, an HtrA polypeptide can be fused to the Fc domain of an immunoglobulin molecule (e.g., an IgG1 molecule) such that active transport of the fusion polypeptide occurs across epithelial cell barriers via the Fc receptor.

An antibody can be, without limitation, a polyclonal, monoclonal, human, humanized, chimeric, or single-chain antibody, or an antibody fragment having binding activity, such as a Fab fragment, F(ab′) fragment, Fd fragment, fragment produced by a Fab expression library, fragment comprising a VL or VH domain, or epitope binding fragment of any of the above. An antibody can be of any type (e.g., IgG, IgM, IgD, IgA or IgY), class (e.g., IgG1, IgG4, or IgA2), or subclass. In addition, an antibody can be from any animal including birds and mammals. For example, an antibody can be a human, rabbit, sheep, chicken, or goat antibody. An antibody can be naturally occurring, recombinant, or synthetic. Antibodies can be generated and purified using any suitable methods known in the art. For example, monoclonal antibodies can be prepared using hybridoma, recombinant, or phage display technology, or a combination of such techniques. In some cases, antibody fragments can be produced synthetically or recombinantly from a gene encoding the partial antibody sequence. An anti-HtrA polypeptide antibody can bind to HtrA polypeptides at an affinity of at least 10⁴ mol⁻¹ (e.g., at least 10⁵, 10⁶, 10⁷, 10⁸, 10⁹, 10¹⁰, 10¹¹, or 10¹² mol⁻¹).

An HtrA polypeptide or antibody can be administered to a mammal in any amount, at any frequency, and for any duration effective to achieve a desired outcome. For example, an HtrA polypeptide can be administered to a mammal under conditions where one or more symptoms of pre-eclampsia or a cardiovascular disease (e.g., one or more symptoms described herein) are prevented or reduced.

An effective amount of an HtrA polypeptide or antibody can be any amount that reduces the severity of pre-eclampsia or a cardiovascular disease without producing significant toxicity to the mammal. If a particular mammal fails to respond to a particular amount, then the amount can be increased by, for example, two-fold. After receiving this higher dose, the mammal can be monitored for both responsiveness to the treatment and toxicity symptoms, and adjustments made accordingly. Various factors can influence the actual effective amount used for a particular application. For example, the frequency of administration, duration of treatment, and route of administration may require an increase or decrease in the actual effective amount administered.

An HtrA polypeptide can be administered once or more than once. The frequency of administration can be any frequency that reduces the severity of pre-eclampsia or a cardiovascular disease without producing significant toxicity to the mammal. For example, the frequency of administration can be from about four times a day to about once a week, or from about once a day to about once a month, or from about once every other day to about once a year. In addition, the frequency of administration can remain constant or can be variable during the duration of treatment. As with the effective amount, various factors can influence the actual frequency of administration used for a particular application. For example, the effective amount, duration of treatment, and route of administration may require an increase or decrease in administration frequency.

An effective duration for administering an HtrA polypeptide or antibody can be any duration that reduces the severity of pre-eclampsia or a cardiovascular disease without producing significant toxicity to the mammal. Thus, an effective duration can vary from several days to several weeks, months or years. Multiple factors can influence the actual effective duration for administering an HtrA polypeptide or antibody. For example, an effective duration can vary with the frequency of administration, effective amount, and route of administration.

Any appropriate method can be used to formulate and administer an HtrA polypeptide or antibody. For example, compositions containing an HtrA polypeptide or antibody can be admixed, encapsulated, conjugated, or otherwise associated with other molecules to assist in uptake, distribution, and/or absorption. Compositions containing an HtrA polypeptide or antibody can contain one or more pharmaceutically acceptable carriers. A pharmaceutically acceptable carrier is a pharmaceutically acceptable solvent, suspending agent, or any other pharmacologically inert vehicle. An HtrA polypeptide or antibody can be administered by a number of methods depending on whether local or systemic treatment is desired and upon the area to be treated. Administration can be, for example, pulmonary (e.g., by inhalation or insufflation of powders or aerosols); oral; or parenteral (e.g., by subcutaneous, intrathecal, intraventricular, intramuscular, or intraperitoneal injection, or by intravenous drip).

The invention will be further described in the following examples, which do not limit the scope of the invention described in the claims.

### EXAMPLES

### Example 1

### Determining the Role of HtrA1 Polypeptide in Pre-Eclampsia

### Materials and Methods

Cell culture: JEG-3 choriocarcinoma cells were cultured in RMPI with L-glutamine (Invitrogen) containing 10% FBS (Invitrogen, Carlsbad, Calif.), 1% penicillin/streptomycin (Invitrogen), and 0.01% fungizone (Invitrogen). The cells were maintained in 100 mm culture dishes at 37° C. under a humidified 5% CO₂/95% air atmosphere. The cells were trypsinized with 0.05% trypsin (Invitrogen) until detached, counted in hemacytometer, and plated in six-well plates (Invitrogen) at a density of 6×10⁵ cells per well. The cells were incubated for 24 hours before transfection with jetPEI (Polyplus-transfection, San Marcos, Calif.) according to manufacturer's protocol.

Western blotting: Western blot analyses of whole cell lysates were performed as described elsewhere (Chien et al., J Clin Invest., 116:1994-2004 (2006)). Whole cell lysates were resolved by 10% SDS-PAGE, and immunoblots were incubated overnight with affinity-purified polyclonal antibodies against HtrA1 (1:1000 in Tris-buffer saline with 0.1% Tween-20 (Sigma, St. Louis, Mo.) and 5% non-fat milk). For gel-loading controls, immunoblots were incubated with monoclonal antibody against β-actin (1:3000). Horse radish peroxidase-conjugated secondary antibodies (Amersham, Piscataway, N.J.) and ECL Detection Reagents (Amersham) were used for immunodetection.

Immunofluorescence microscopy: JEG-3 cells were transfected for 24 hours with a vector, or with a vector containing a nucleic acid encoding a protease mutant HtrA1 polypeptide, or a wild-type HtrA1 polypeptide. The transfected cells were trypsinzed and re-seeded over glass coverslips in 35 mm dishes. The cells were then fixed in 4% paraformaldehyde for 10 minutes at 25° C., permeabilized with Tris-buffer saline with 0.5% Tween-20 (TBST), and blocked with 5% non-fat milk in TBST. The coverslips were incubated with affinity-purified HtrA1 antibodies (1:100) for one hour at 25° C., washed in TBST, and visualized using FITC-conjugated secondary antibody (Sigma). The cells were also counter-stained with TRITC-conjugated Phalloidin for F-actin.

Scratch assay: JEG-3 cells were plated in 24-well plates (50,000 cells/well) and transfected with a vector containing a nucleic acid encoding a GFP polypeptide, a protease mutant HtrA1 (SA) polypeptide, or a wild-type HtrA1 (WT) polypeptide using jetPEI transfection reagent. Forty-eight hours after transfection, when monolayer culture was achieved, scratches were made in the monolayers using 200 μL pipette tips. Two positions along the scratch (one scratch/well) were marked with a black marker and photographed at 0 hour and 24 hours after the scratches were made using a SPOT camera (Diagnostic Instruments, Sterling Heights, Mich.). Gaps created by scratches at 0 and 24 hours were measured using SPOT software, and percent gap filled was calculated. Each group contained four replicates and eight measurements.

Transwell migration assay: JEG-3 cells were plated in 35 mm dishes (500,000 cells/well). The cells were transfected 24 hours later with a vector containing a nucleic acid encoding a GFP polypeptide, SA HtrA1 polypeptide or WT HtrA1 polypeptide. The cells were then trypsinized, resuspended in serum-free medium, and counted, and 20,000 cells were seeded into Transwell chambers (Millipore, Billerica, Mass.). Growth medium was added to the lower chambers. Twenty-four hours later, cells were fixed in 4% paraformaldehyde, permeabilized in TBST, stained with propidium iodide, and visualized by fluorescence microscopy. Five 10× fields/sample were counted, and averages were calculated. Each experiment contained triplicate samples.

Matrigel invasion assay: JEG-2 cells were transfected as described above, trypsinized, and re-seeded into Matrigel-coated chambers (BD Biosciences, San Jose, Calif.) at the density of 40,000 cells/chamber. The cells were fixed 24 hours later, stained with propidium iodide, and visualized by fluorescence microscopy. Five 10× fields/samples were counted, and averages were calculated. Each experiment contained triplicate samples.

Patient selection: The diagnosis of severe pre-eclampsia was based on strict American College of Obstetricians and Gynecologists (ACOG) criteria. Patients with a history of chronic hypertension, pregestational diabetes mellitus, thrombophilias, clinical evidence of infection, autoimmune disease, and antiphospholipid antibody syndrome were excluded.

Samples: Twenty-seven singleton placentas, at different gestational age ranges in the third trimester, were obtained from normotensive patients, controls (n=13) and severe pre-eclampsia patients (n=14). Different gestational age ranges were evaluated in the normotensive patients and those patients affected with severe pre-eclampsia.

Normotensive: 28-31 6/7 weeks gestation (n=4), 32-36 6/7 weeks gestation (n=5), and >37 weeks gestation (n=4). Severe pre-eclampsia: n=3, n=7, and n=4, respectively. The collected samples were immediately fixed in 10% formalin for immunohistochemistry.

Immunohistochemistry: Immunohistochemistry Select EDTA buffer (Chemicon, Temecula, Calif.) was used for antigen retrieval before immunohistochemical staining Placentas were pre-blocked with ChemMate (Ventana Medical Systems, Tucson, Ariz.) and incubated with 1:200 diluted affinity-purified anti-HtrA1 antibodies in ChemMate. Placental sections were washed three times in PBS containing 0.2% Tween 20 and incubated with anti-rabbit immunoglobulin antibodies conjugated to horseradish peroxidase. After washing the slides three times in PBS-Tween 20, immunocomplexes were visualized with the DAB Substrate Kit (Zymed, Carlsbad, Calif.).

Placental Analysis The level of HtrA1 expression was qualitatively rated via light microscopy by a placental pathologist who was blinded to the clinical history of the patient. A score of 0 (no positive cells), 1 (minimal staining), 2 (moderate staining), and 3 (marked staining) was assigned. Three slides per specimen, on average, were evaluated. The Student's t-test was used to compare the means of the rating score between the groups. Statistical significance was defined as P<0.05 at α=0.05.

### Results

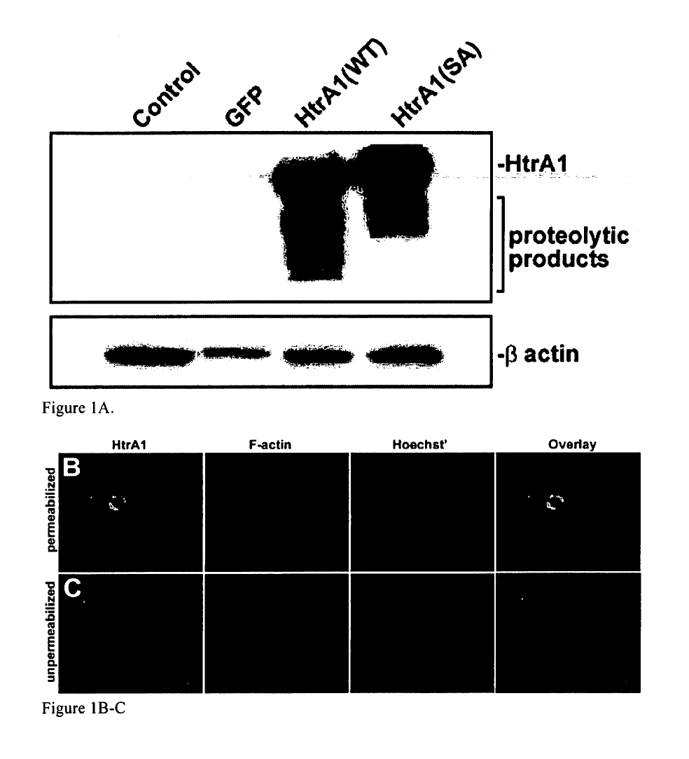

HtrA1 polypeptide expression in JEG-3 trophoblasts: Western blotting of JEG-3 cell lysates revealed that HtrA1 polypeptide was minimally expressed in JEG-3 trophoblasts. After transient transfection with a vector containing a nucleic acid encoding a GFP polypeptide, a wild-type HtrA1 (WT) polypeptide, or a protease mutant HtrA1 (SA) polypeptide, Western blot analysis indicated over-expression of HtrA1 polypeptide in cells transfected with a vector encoding a WT or SA HtrA1 polypeptide (FIG. 1A). Immunofluorescence microscopy revealed significant polypeptide expression within and outside the cytoplasm (FIGS. 1B and C, respectively). Immunofluorescence analysis of unpermeabilized JEG-3 cells showed diffuse localization of HtrA1 polypeptide around the cells. When cells were permeabilized, HtrA1 polypeptide staining was observed around the nucleus.

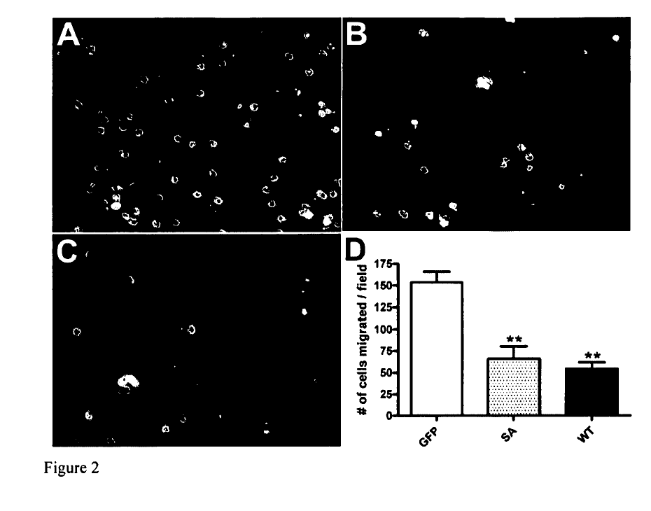

Effect of HtrA1 polypeptide expression on trophoblastic JEG-3 cell transwell migration: It was determined whether aberrant expression of HtrA1 polypeptide affects trophoblastic JEG-3 migration and invasion. JEG-3 trophoblasts were transfected as described above, and two separate migration assays were performed. Transwell migration assays revealed significantly attenuated migration of JEG-3 cells following aberrant expression of the WT or SA HtrA1 polypeptide compared to the GFP control. There was a 60% decrease in transwell migration in JEG-3 cells transfected with both forms of HtrA1 polypeptide (P<0.01) compared to control cells transfected with a vector containing a nucleic acid encoding a GFP polypeptide. No statistically significant difference was observed between cells transfected with a vector encoding a WT or SA HtrA1 polypeptide (FIG. 2).

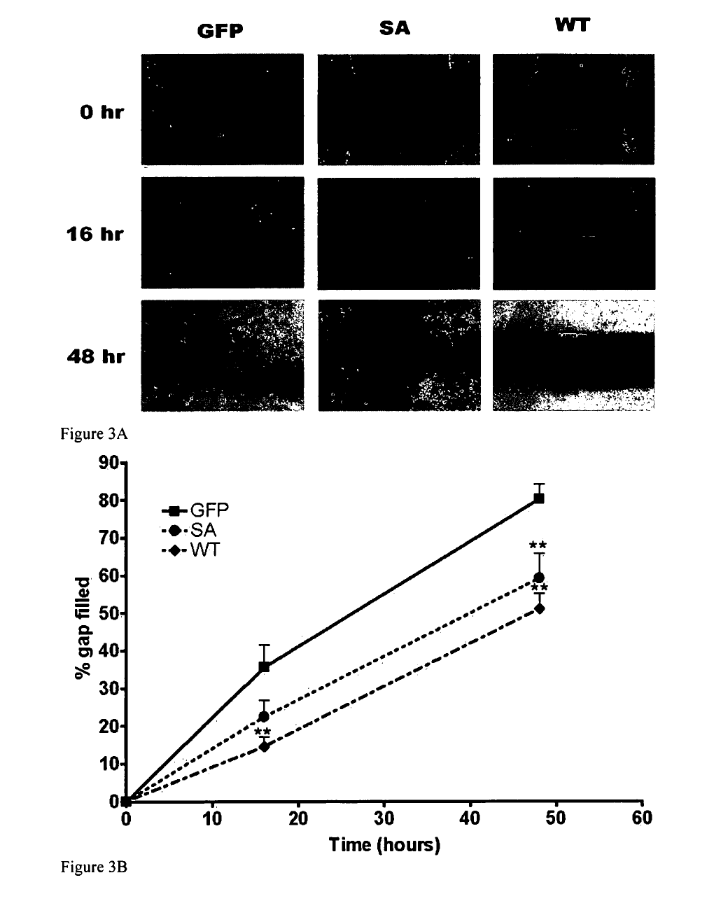

Effect of HtrA1 polypeptide expression on trophoblastic JEG-3 cell migration: To further investigate the effect of HtrA1 polypeptide expression on JEG-3 cell migration, scratch assays were performed on transfected cells grown to monolayer culture. Closure of the gap was evaluated serially at three time points: 0-hr, 16-hr, and 48-hr. Photomicrographs were taken at these time points (FIG. 3A). At 16 hours, a statistically significant decrease in gap closure was observed in cells transfected with a vector encoding a WT HtrA1 polypeptide, but not in cells transfected with a vector encoding an SA polypeptide, as compared to cells transfected with a vector encoding a GFP polypeptide (P<0.01; FIG. 3B). At 48 hours, there was a statistically significant decrease in gap closure in cells transfected with a vector encoding a WT or SA HtrA1 polypeptide as compared to cells transfected with a vector encoding a GFP polypeptide (P<0.01; FIG. 3B). There was no statistically significant difference between cells transfected with a vector encoding a WT or SA HtrA1 polypeptide at either the 16 or 48 hour timepoint.

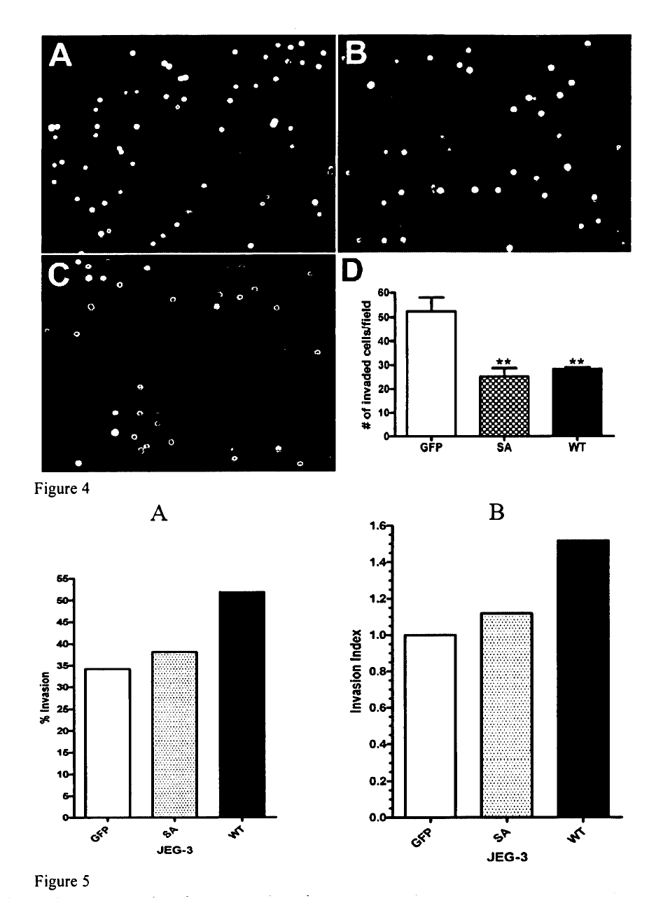

Effect of HtrA1 polypeptide expression on trophoblastic JEG-3 cell invasion: It was investigated whether forced expression of HtrA1 would suppress invasiveness of JEG-3 cells. Exogenous expression of WT or SA HtrA1 polypeptide resulted in a marked decrease in JEG-3 cell invasion through a Matrigel coated membrane compared to control cells transfected with a vector containing a nucleic acid encoding a GFP polypeptide (FIG. 4).

Decrease in JEG-3 cell invasion due to inhibition of cell migration: The invasion index was calculated to determine whether the diminished invasion observed in cells transfected with a vector containing a nucleic acid encoding a WT or SA HtrA1 polypeptide was associated with attenuated migration. Although a decrease was observed in the total number of invading cells, an increase was observed in percent invasion (FIG. 5A) and invasion index (FIG. 5B) associated with WT HtrA1 polypeptide expression. These data suggest that a decrease in cell invasion associated with HtrA1 polypeptide expression is due to a diminished migratory capacity of JEG-3 cells.

Aberrant expression of HtrA1 polypeptide in placentas collected from pregnancies complicated by severe pre-eclampsia: Immunohistochemical analysis of HtrA1 polypeptide expression in placental samples collected from normal and pre-eclamptic pregnancies indicated that HtrA1 polypeptide expression was detected in both the villous trophoblasts and extravillous cytotrophoblasts in all samples. Patient characteristics are shown in Table 1.

TABLE 1
Patient characteristics

| | | | | | | |
|---|---|---|---|---|---|---|
| Gestational | | | | VT staining | EVT staining | Indication for |
| age | ROM >18 hr | Labor | Mode of delivery | score | score | delivery |

Normotensive

| | | | | | | |
|---|---|---|---|---|---|---|
| 39 + 5 | N | Y | NSVD | 1.5 | 2.5 | |
| 39 + 3 | N | N | Repeat C/S | 0 | 2 | |
| 39 + 1 | N | N | Elect C/S | 1 | 2 | |
| 37 + 3 | N | Y | Repeat C/S | 1 | 2 | |
| 36 + 0 | N | Y | NSVD | 2 | | PTL |
| 35 + 3 | N | Y | NSVD | 3 | 2.5 | PPROM |
| 35 + 0 | N | Y | Prim C/S | 0 | 3 | PTL, active genital herpes |
| 34 + 5 | N | N | Prim C/S | 1 | 3 | Vasa previa |
| 34 + 1 | N | Y | Forceps | 1 | 2.5 | PTL |
| 31 + 6 | N | N | Prim C/S | 1 | 2 | Abruption |
| 31 + 4 | N | N | Repeat C/S | 1 | | Abruption |
| 31 + 2 | N | Y | Prim C/S | 2 | 2 | Lung cancer in mother |
| 29 + 1 | N | N | Repeat C/S | 1.5 | 1.5 | Uterine rupture |

Severe Preeclampsia

| | | | | | |
|---|---|---|---|---|---|
| 38 + 5 | N | Y | NSVD | 2.5 | |
| 37 + 6* | N | Y | NSVD | 1 | 2.5 |
| 37 + 2 | N | Y | NSVD | 2.5 | |
| 37 + 1* | N | Y | NSVD | 0 | 2 |
| 36 + 3 | N | N | Prim C/S | 2.5 | 2 |
| 35 + 6 | N | Y | NSVD | 3 | 3 |
| 35 + 3 | N | Y | NSVD | 3 | 2.5 |
| 34 + 2 | N | Y | NSVD | 2 | 3 |
| 33 + 3 | N | Y | NSVD | 3 | 2.5 |
| 32 + 0 | N | Y | NSVD | 3 | 3 |
| 31 + 4 | N | N | Prim C/S | 3 | 3 |
| 30 + 3 | N | N | Repeat C/S | 2 | 2 |
| 29 + 3 | N | Y | NSVD | 2.5 | 3 |

*HELLP as only criteria for severe preeclampsia
WGA = weeks gestational age, NSVD = normal spontaneous vaginal delivery, Elect C/S = elective cesarean, Prim C/S = primary cesarean, PTL = preterm labor, PPROM = preterm premature rupture of membranes
VT = villous trophoblast, EVT = extravillous trophoblast

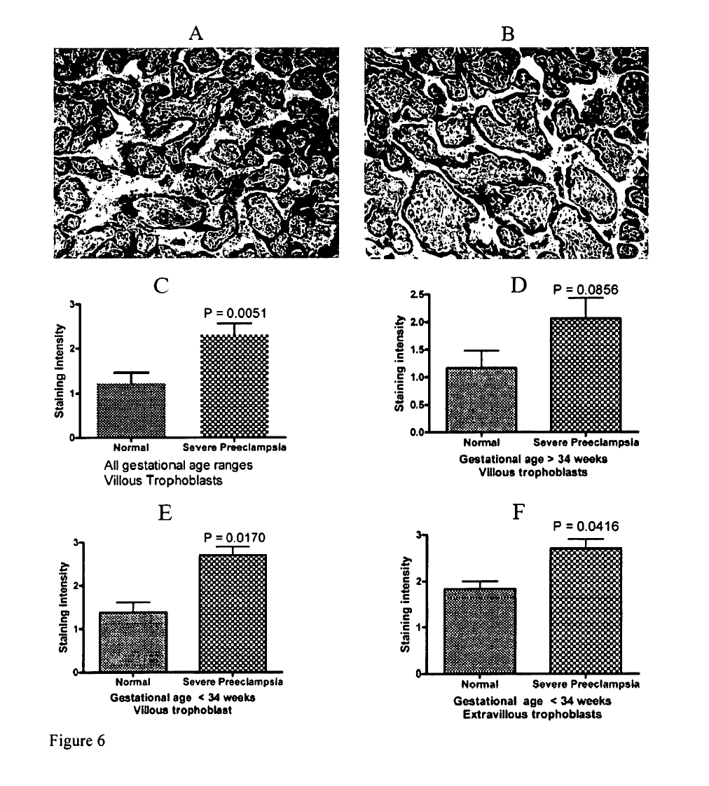

Aberrant expression of HtrA1 polypeptide in villous trophoblast was observed in samples collected from severe pre-eclampsia (FIG. 6A) compared to samples collected from gestational age-matched normal pregnancy (FIG. 6B). Immunohistochemical analysis indicated higher levels of HtrA1 polypeptide expression in placental tissue from pregnancy complicated by pre-eclampsia as compared to placental tissue from normal pregnancy (FIGS. 6A and 6B). Statistical analysis of HtrA1 polypeptide staining in villous trophoblasts from normal and severe pre-eclamptic pregnancies at all gestational age ranges indicated an aberrant increase in HtrA1 polypeptide expression in severe pre-eclampsia (FIG. 6C). HtrA1 polypeptide staining in villous trophoblasts after 34-weeks gestational age also showed an increase in staining intensity in severe pre-eclamptics compared to gestational-age matched normotensive controls, although the difference was not statistically significant (FIG. 6D). HtrA1 polypeptide staining in villous trophoblasts prior to 34-weeks gestational age, however, showed a statistically significant increase in HtrA1 polypeptide staining in severe pre-eclamptics compared to their gestational-age matched controls (FIG. 6E). Moreover, extravillous cytotrophoblasts from severe pre-eclamptic placentas prior to 34-weeks gestational age also showed an aberrant increase in HtrA1 polypeptide staining (FIG. 6F). These data suggest that aberrant expression of HtrA1 can be associated with pre-eclamptic pregnancies.

Staining was noted primarily in the cytoplasm, and some nuclear staining was also observed. Interestingly, positive, albeit minimal, HtrA1 polypeptide staining was also observed in the amnion, endothelium of vessels, and Wharton's jelly. There was no staining of HtrA1 polypeptide in smooth muscle.

No HtrA1 polypeptide expression was observed in the villous trophoblasts from two of the specimens from the severe preeclamptic group. A difference in these patients was that their diagnosis of severe preeclampsia was based solely on HELLP syndrome. Also, their disease developed after 37 weeks gestational age. The other patients had a gestational age of less than 37 weeks and had elevated blood pressures along with HELLP syndrome, proteinuria, or other signs or symptoms of severe preeclampsia. There was moderate expression in the villous trophoblasts of two and marked expression in one of the samples from the normotensive group. The histology from these placentas was normal.

### Example 2

### Detecting Serum HtrA1 Polypeptide Levels

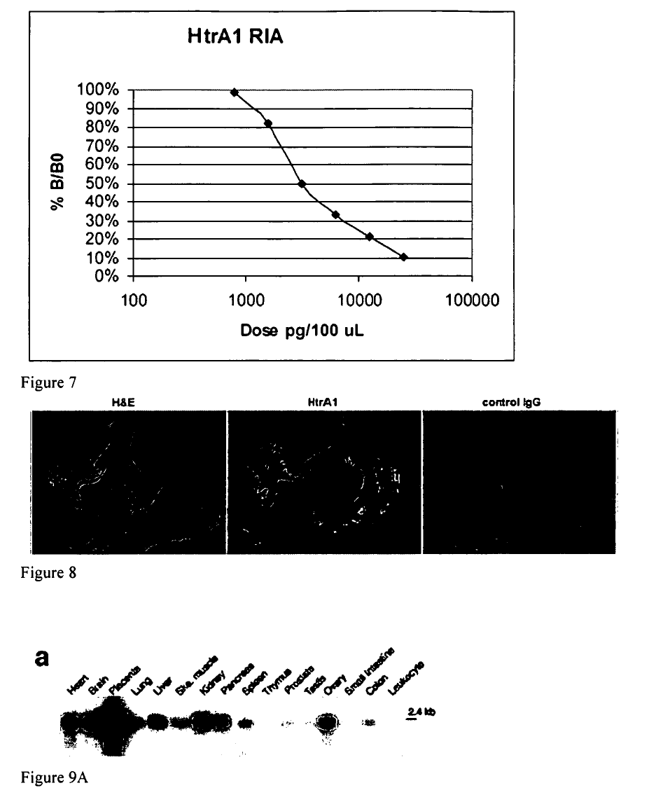

Purified HtrA1 polypeptide was radiolabeled with ¹²⁵I and used in a competitive radioimmunoassay (RIA). Known amounts of unlabeled HtrA1 polypeptide were used as standard controls, and diluted serum samples were used as unknown test samples. A standard curve generated using known standards is presented in FIG. 7. The RIA also was used to detect amounts of HtrA1 polypeptide in human serum from eight first trimester pregnancies (Table 2).

TABLE 2
HtrA1 polypeptide levels in human
serum measured by radioimmunoassay

| | | | | | ng/100 μL | ng/mL |
|---|---|---|---|---|---|---|
| Description | Dilution | cpm | Av cpm | % B/B0 | Curve value | Final |
| Sample 3 | 1:2.5 | 2909 | 2883 | 87% | 1.3 | 33 |
| | (40 μL) | 2856 | | | | |
| Sample 4 | 1:2.5 | 2867 | 2854 | 85% | 1.4 | 35 |
| | | 2840 | | | | |
| Sample 5 | 1:2.5 | 2882 | 2954 | 90% | 1.15 | 29 |
| | | 3025 | | | | |
| Sample 6 | 1:2.5 | 3032 | 2983 | 91% | 1.1 | 28 |
| | | 2934 | | | | |
| Sample 7 | 1:2.5 | 2977 | 3032 | 94% | 1 | 25 |
| | | 3086 | | | | |
| Sample 8 | 1:2.5 | 3228 | 3099 | 97% | 0.88 | 22 |
| | | 2970 | | | | |
| Sample 9 | 1:2.5 | 3094 | 3067 | 95% | 0.95 | 24 |
| | | 3040 | | | | |
| Sample 10 | 1:2.5 | 2939 | 2934 | 89% | 1.2 | 30 |
| | | 2929 | | | | |

Four monoclonal antibodies and polyclonal antibodies against HtrA1 polypeptide were generated for use in sandwich ELISAs.

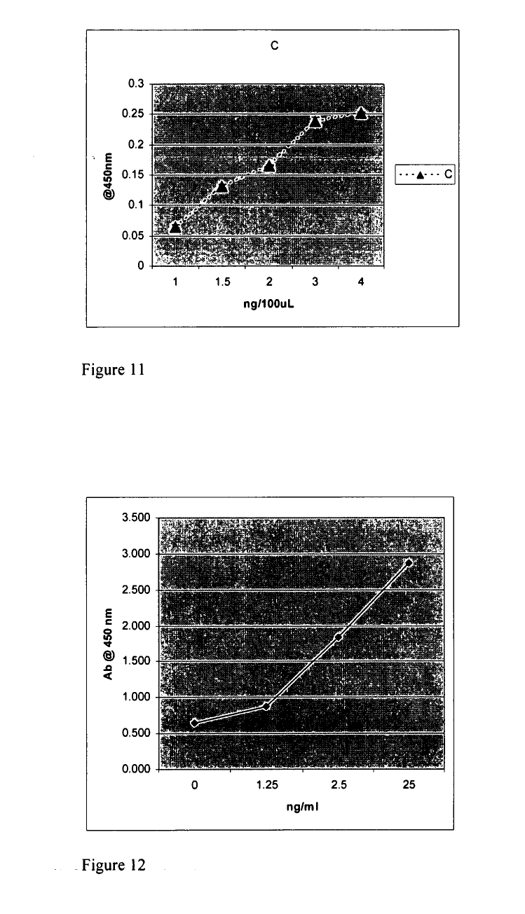

Known amounts of HtrA1 polypeptide were applied to each well in 96-well plate. Then, primary rabbit polyclonal antibodies against HtrA1 polypeptide was used with anti-rabbit antibodies labeled with HRP to detect the quantity (ng/mL) of HtrA1 polypeptide in each well (FIG. 11). These results demonstrate that the anti-HtrA1 polypeptide antibodies can detect ng quantities of HtrA1 polypeptide.

To perform a sandwich ELISA, wells are coated with two monoclonal HtrA1 antibodies to capture serum HtrA1 polypeptide. After washing the wells with PBS to remove unbound antibody, 100 μL of serum are applied to each well and incubated for 30 minutes at room temperature. After washing the wells with PBS, polyclonal antibodies are added to each well, and immunocomplexes are detected using anti-rabbit HRP and the TMB substrate kit (Pierce, Rockford, Ill.). Known amounts of recombinant HtrA1 polypeptide are added into separate wells and serve as standards.

To demonstrate the sensitivity of a sandwich ELISA procedure to ng quantities of HtrA1 polypeptide, known amounts of HtrA1 polypeptide were applied to each well in 96-well plate. Then, after washing to remove unbound HtrA1 polypeptide, polyclonal HtrA1 antibodies were added to each well. Anti-rabbit antibodies labeled with HRP were used to detect the quantity of HtrA1 polypeptide in each well (FIG. 12). These results demonstrate that a sandwich ELISA procedure can be used to detect ng quantities of HtrA1 polypeptides.

Indirect ELISAs are performed by applying 100 μL of a serum sample to each well of an ELISA plate and incubating the plate overnight at 4° C. Known amounts of recombinant HtrA1 polypeptide are added into separate wells and serve as standards.

After washing to remove unbound HtrA1 polypeptide, polyclonal antibody is applied to each well, and immunocomplexes are detected using anti-rabbit HRP and the TMB substrate kit (Pierce).

### Example 3

### Screening for HtrA1 Antibodies

Screening is performed using the random peptide library (JPT Peptide Technologies, Springfield, Va.), which contains four peptide microarrays comprising a total of 1,536 fluorescently labeled polypeptides containing 21,504 potential cleavage sites. Reporter fluorescence dye (fluorophore) is separated from the quencher by a linker region containing 21,504 potential protease-sensitive cleavage sites. The cleavage of specific sites by HtrA1 removes the quencher away from fluorophore, allowing the fluorophore to fluoresce, thereby identifying specific cleavage sites of HtrA1 polypeptide.

After identifying specific substrates for HtrA1 polypeptide by screening the random peptide library, wells of an assay plate are coated with different monoclonal antibodies, which are allowed to capture recombinant HtrA1 polypeptides. Four monoclonal antibodies against HtrA1 polypeptide were generated by immunizing mice with bacterial-purified recombinant HtrA1 polypeptides 141-480. After washing the wells to remove unbound HtrA1 polypeptides, specific substrate is added into wells, and HtrA1 protease activity is monitored. If a specific monoclonal antibody disrupts HtrA1 polypeptide activity, minimal HtrA1 polypeptide activity is detected in the well coated with such antibody. This technique allows specific monoclonal antibodies that block HtrA1 protease activity to be identified. To demonstrate the ability of these monoclonal antibodies to capture HtrA1 polypeptide, a sandwich ELISA is carried out with monoclonal antibodies as a capture mechanism and polyclonal antibodies as a detection mechanism.

### Example 4

### Evaluating HtrA1-Targeted Therapy

A rat model of preeclampsia is established by administering desoxycorticosterone acetate (DOCA) to pregnant rats being provided with 0.9% saline as drinking water (Ianosi-Irimie et al., Clinical and Experimental Hypertension, 8:605-617 (2005)). The rats develop preeclampsia-like syndrome and are randomly assigned into two groups: a test group that receives vaginal delivery of HtrA1 antibody or immunization against HtrA1 polypeptide, and a control group that receives vehicle controls. Systolic blood pressure is monitored by the tail-cuff method, and urine and blood analyses are performed. The pyrogallol red method (Total Protein Kit, Micro Pyrogallol Red Method, Sigma) is used for urine protein analysis. The Beckman Creatinine Analyzer 2 and the creatinine reagent kit (Beckman Coulter) are used for creatinine analysis. An HtrA1 ELISA is used to determine serum levels of HtrA1 polypeptide. For the rats receiving HtrA1 immunization, the presence of antibodies is detected by ELISA. At 20 days of pregnancy, the animals are euthanized, and litter size and weight are recorded to determine the effect of HtrA1-targeted therapy on pre-eclampsia.

### Example 5

### Determining the Role of HtrA1 Polypeptide in Atherosclerosis

To investigate whether HtrA1 polypeptide expression is localized to atherosclerotic plaques, aorta samples from ApoE^{-/-} mice on 20 weeks of high fat-high cholesterol diet were collected and stained with an anti-HtrA1 antibody. H&E staining indicated the presence of atherosclerotic plaque in these mice (FIG. 8). HtrA1 staining (brown staining) was localized to these plaques. Brown staining was not observed in samples stained with a control IgG antibody, indicating the specificity of the HtrA1 antibody in the immunohistochemical technique.

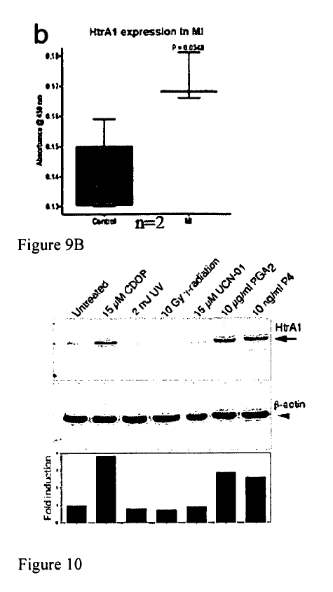

To analyze the tissue-specific expression of HtrA1 polypeptide, Northern blot analysis was performed using RNA samples isolated from various normal tissues. The results indicated that HtrA1 is expressed in heart tissue as well as in ovary, placenta, and brain tissue (Chien et al., Oncogene, 23(8):1636-44 (2004); FIG. 9A). Analysis of circulating HtrA1 polypeptide from rats with experimentally-induced myocardial infarction showed that HtrA1 polypeptide levels are elevated in rats with myocardial infarction compared to controls (FIG. 9B).

To determine whether HtrA1 polypeptide expression can be regulated by various agents including sex hormones, an ovarian cell line, OSEtST, was treated with various agents for 24 hours, and immunoblot analysis was performed on whole cell lysates. The results indicated that HtrA1 polypeptide expression is regulated by progesterone (FIG. 10).

These results indicate a pathophysiologic role of HtrA1 polypeptide in atherosclerosis.

The clinical value of HtrA1 polypeptide in prognosis of coronary artery disease and risk stratification of acute coronary syndrome is evaluated. Circulating HtrA1 polypeptide levels are correlated with various cardiac presentations, and with clinical outcomes (death, ACS, or revascularization) in patients with chronic CAD. Circulating HtrA1 polypeptide levels are also evaluated as a marker for risk stratification of patients with chest pain.

The prognostic value of circulating HtrA1 polypeptide in patients with CAD is evaluated. Samples collected from 333 subjects enrolled in a study (Elesber et al., Eur. Heart J., 27(14):1678-84 (2006)) are used. Among these participants, 27 patients had an acute myocardial infarction (MI), 110 had unstable angina, 86 had no significant CAD, and 110 had chronic CAD (diameter stenosis of ≧50% in at least one diameter). Seven participants from chronic CAD were excluded from the study due to presence of hypertrophic cardiomyopathy or significant inflammatory conditions. Other exclusion criteria included congenital heart disease, current infection, vasculitis, or rheumatic disease. MI, stable angina, and unstable angina were defined as described elsewhere (Elesber et al., Eur. Heart J., 27(14):1678-84 (2006)). For risk stratification in patients with angina, 59 patients who presented with chest pain in the emergency department were enrolled in the study (Elesber et al., Int. J. Cardiol. (2006)), and the diagnosis of presence or absence of an acute coronary syndrome was made. Known risk factors, such as family history, age, smoking, hypertension, diabetes, and hyperlipidemia, were abstracted for each patient.

Assays for circulating HtrA1 polypeptide are performed by ELISA. A polyclonal HtrA1 antibody is used to capture HtrA1, and a combination of monoclonal antibodies is used for detection. Known concentrations of recombinant human HtrA1 are used as standards to calculate circulating concentrations of HtrA1 polypeptide.

The statistical analysis is carried out independently by a statistician. Independent variables (age, family history of CAD, smoking, hypertension, diabetes, hyperlipidemia, hypercholesterolemia, body mass index, EF, extent of atherosclerosis, C-reactive protein, prior medical or surgical treatment for chronic CAD or myocardial infarction, HtrA1, troponin, and MB CPK levels) are analyzed either as continuous, binary, or categorical (e.g., tertiles, quartiles). Dependent or outcome variables are: MI, unstable angina, stable angina (binary outcomes); survival/revascularization after diagnosis (time-to-event outcomes). A Student's t-test, Pearson's χ2-statistic, or the Mann-Whitney rank sum test is performed to compare group differences. A Pearson's χ2-statistic or the Mann-Whitney rank sum test is used to analyze significant predictors of binary outcomes at univariate analysis, while a logistic regression model is employed to assess independent predictors at multivariate analysis. Kaplan-Meier analysis is performed on survival outcome information using log-rank test for significance. Cox proportional hazards models are performed to estimate the unadjusted and proportional hazard ratios for outcome analysis. P<0.05 is considered significant.

The association between genetic polymorphisms in HtrA1 and atherosclerotic vascular disease is investigated. HtrA1 genetic polymorphisms are identified in 400 African-American and 400 non-Hispanic white participants. HtrA1 AA genetic polymorphism is correlated with coronary artery calcification, and with survival outcome. Genetic contributions of complement factor H and HtrA1 in atherosclerosis are evaluated.

HtrA1 genetic polymorphisms are identified in 400 African-American and 400 non-Hispanic white participants from the GENOA (Genetic Epidemiology Network of Atherosclerosis) study as part of the Family Blood Pressure Program (FBPP). The FBPP study includes 11,357 subjects and contains >120 measured and derived phenotypic variables (Hypertension, 39(1):3-9 (2006)). Four hundred subjects with adverse outcome (myocardial infarction, acute coronary syndrome, revascularization, or death), and 400 control subjects without adverse outcome, are selected from the two ethnic cohorts. HtrA1 rs11200638 and CFH rs1061170 (Y402H) genotypes are analyzed using Taqman assays as described elsewhere (Magnusson et al., PLoS Med., 3(1):e5 (2006)).

Statistical Analysis: The χ2 statistic for HtrA1 polymorphisms is performed to assess evidence for association with coronary calcification and survival outcome. Odds ratios and 95% confidence intervals are calculated to estimate risk size for the heterozygotes and homozygotes for the risk alleles. Two-locus analyses are performed for the CFH rs1061170 (Y402H) polymorphisms and HtrA1 rs11200638 polymorphisms. A contingency table based on case-control status and two-locus genotype combination is constructed. The two-locus genotype combinations across for CFH and HtrA1 include TT/GG, TT/AG, TT/AA, CT/GG, CT/AG, CT/AA, CC/GG, CC/AG, and CC/AA. The two-locus 9×2 contingency table is tested with a χ2 statistic. Odds ratios and 95% confidence intervals for each genotypic combination are compared to the TT/GG homozygosity. When the risk genotypes is identified, population attributable risks (PAR) are calculated using Levin's formula (Acta Unio Int. Contra Cancrum, 1953, 9(531)). Additional cardiovascular risk factors are used in the multivariate analyses to determine whether specific polymorphisms have independent prognostic value.

The genetic contributions of HtrA1 polymorphisms in cardiovascular disease risk are investigated in early menopausal women. HtrA1 polymorphisms are genotyped in the post-menopausal women cohort from the Kronos Early Estrogen Prevention Study (KEEPS), and different polymorphisms are correlated with cardiovascular events. HtrA1 polymorphisms also are genotyped in the post-menopausal women cohort from the Study of Tamoxifen and Raloxifene (STAR), and different polymorphisms are correlated with cardiovascular events. A two-locus analysis of HtrA1 polymorphisms and CYP2D6 polymorphisms is performed to determine the genetic interactions between these two genes in modifying clinical outcome associated with cardiovascular disease in two study cohorts.

The genotypes of HtrA1 polymorphisms in samples from the KEEPS double-blinded study cohort of 720 women are identified using a Taqman assay. Imaging of the carotid and coronary arteries is performed, and carotid intimal medial thickness (IMT), and coronary calcification are determined, in the KEEPS study. These endpoints, as well as adverse clinical outcome defined as incidence of death due to myocardial infarction, acute coronary syndrome, revascularization, thrombosis, and stroke, are used to determine whether HtrA1 genetic polymorphisms are associated with particular cardiovascular endpoints. Because KEEPS is designed to document both protective and risk factors for heart disease, family history, metabolic syndrome, diabetes mellitus, history of smoking, hypertension, and hyperlipidemia are available and can be abstracted from the study cohort to perform multivariate analysis to determine if any one of these risk factors influences the contributions of HtrA1 polymorphism towards development of adverse clinical outcome during the trial.

Samples obtained from the STAR trial are genotyped. The STAR trial enrolled 19,747 women, and 19,471 women completed the study. Eighty-seven of 9,726 women in the tamoxifen group, and 65 of 9,745 women taking raloxifene, had a deep vein thrombosis. Fifty-four of 9,726 women taking tamoxifen, and 35 of 9,745 women taking raloxifene, developed pulmonary embolisms. Fifty-three of 9,726 women in the tamoxifen group, and 51 of 9,745 women in the raloxifene group, had a stroke during the trial. Samples corresponding to these adverse cases as well as age- and treatment-matched control samples without any adverse effect in the same study are used in the genotyping. These adverse outcomes are used as the endpoints to determine whether a specific HtrA1 polymorphism is associated with these outcomes.

The χ2 statistic for HtrA1 polymorphisms is performed to assess evidence for association with coronary disease progression, carotid IMT, coronary calcification, thrombosis, stroke, and survival outcome in KEEPS study. Odds ratios and 95% confidence intervals are calculated to estimate risk size for the heterozygotes and homozygotes for the risk alleles. Similar analyses are done using samples from the STAR trial. Two-locus analyses are performed for the CYP2D6 polymorphisms and HtrA1 rs11200638 polymorphisms. A contingency table based on case-control status and two-locus genotype combination is constructed. The two-locus genotype is analyzed by the two-locus contingency table with a χ2 statistic. Odds ratios and 95% confidence intervals for each genotypic combination are compared to the wild-type homozygosity. When the risk genotypes are identified, population attributable risks (PAR) are calculated using Levin's formula (Acta Unio Int. Contra Cancrum, 1953, 9(531)). Additional cardiovascular risk factors are used in the multivariate analyses to determine whether specific polymorphisms have independent prognostic value.

### Example 6

### Immunizing Mice with Recombinant HtrA1 Polypeptide

Mice are immunized with recombinant human HtrA1 polypeptide to generate monoclonal antibodies directed against human HtrA1 polypeptide.

APOE^{-/-} or APOE^{-/-} & HtrA1 WT transgenic mice are separated into two groups: control and immunized cohorts. Mice in the control cohort receive Freund's complete adjuvant (Difco, Becton Dickinson, Sparks, Md.). Mice in the immunized cohort receive 0.1 mg of recombinant HtrA1 polypeptide emulsified in Freund's complete adjuvant by subcutaneous and intra-peritoneal injections. Fourteen days after initial immunization, mice in the immunized cohort receive 0.1 mg of HtrA1 polypeptide as a booster. Twenty-eight days after initial immunization, the mice are bled and screened for their immune responsiveness to HtrA1 polypeptide by ELISA. For ELISA, 50 ng/100 μL of recombinant HtrA1 polypeptide is added into each well of a 96 well plate (Pro-Bind, Becton Dickinson) and incubated overnight in Coating Buffer (50 mM NaHCO₃, pH 9.6). Wells are then washed with PBS, blocked with 3% BSA, and incubated for one hour at room temperature with 1:10 diluted serum collected from mice. After washing the wells to remove unbound antibodies, 100 μL of goat anti-mouse HRP is added into each well, and incubated for 30 minutes at room temperature. After thoroughly washing the wells, immunocomplexes are visualized using the TMB substrate kit. Immunoresponse from the mice is correlated with disease progression as determined by the numbers and the size of atherosclerotic plaques.

Immunization of APOE^{-/-} mice and APOE^{-/-} & HtrA1 transgenic mice and production of antibodies against HtrA1 polypeptide in the mice can inhibit atherosclerotic plaque growth and progression when compared to non-immunized mice.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of identifying pre-eclampsia in a mammal, said method comprising (a) determining whether or not a mammal contains an elevated level or activity of an HtrA polypeptide, and (b) classifying said mammal as having pre-eclampsia when said elevated level or activity is present.

2. The method of claim 1, wherein said mammal is a human.

3. The method of claim 1, wherein said HtrA polypeptide is an HtrA1 polypeptide.

4. The method of claim 1, wherein said level or activity is a serum level or activity.

5. The method of claim 1, wherein said determining step comprises using a radioimmunoassay, an ELISA, or a proteolytic colorimetric assay.

6. A method of identifying a cardiovascular disease in a mammal, said method comprising (a) determining whether or not a mammal contains an elevated level or activity of an HtrA polypeptide, and (b) classifying said mammal as having a cardiovascular disease when said elevated level or activity is present.

7. The method of claim 6, wherein said mammal is a human.

8. The method of claim 6, wherein said cardiovascular disease is atherosclerosis or acute coronary syndrome.

9. The method of claim 6, wherein said HtrA polypeptide is an HtrA1 polypeptide.

10. The method of claim 6, wherein said level or activity is a serum level or activity.

11. The method of claim 6, wherein said determining step comprises using a radioimmunoassay, an ELISA, or a proteolytic colorimetric assay.

12. A method of predicting the susceptibility of a mammal to develop pre-eclampsia, said method comprising (a) determining whether or not a mammal has an HtrA polymorphism, and (b) classifying said mammal as being susceptible to develop pre-eclampsia when said HtrA polymorphism is present.

13. The method of claim 12, wherein said mammal is a human.

14. The method of claim 12, wherein said HtrA polymorphism is an HtrA1 polymorphism.

15. The method of claim 12, wherein said HtrA1 polymorphism is the single nucleotide polymorphism rs11200638.

16-38. (canceled)
